(19) United States
(12) Patent Application Publication
(10) Pub. No.: US 2008/0287552 A1
(43) Pub. Date: Nov. 20, 2008

(54) Process for Obtaining an Active Ingredient for Enhancing Cutaneous Mechanical Strength, Active Ingredient and Compositions

(75) Inventor: Jean Paufique, Objat (FR)

Correspondence Address: YOUNG & THOMPSON, 209 Madison Street, Suite 500, ALEXANDRIA, VA, 22314, US

(73) Assignee: SOCIETE INDUSTRIESLLE LIMOUSINE D'APPLICATION (SILAB) LIEUDIT MADRIAS, OBJAT (FR)

(21) Appl. No.: 12/093,716

(22) PCT Filed: Nov. 17, 2006
(86) PCT No.: PCT/FR06/51188

§ 371 Date: Jun. 23, 2008

(30) Foreign Application Priority Data
Nov. 18, 2005 (FR) 05-53497

(51) Int. Cl. A61K 47/26; C12P 1/00; A61P 17/00

(52) U.S. Cl. 514/777; 435/41

(57) ABSTRACT

The object of the invention is a process for obtaining an active ingredient for increasing the mechanical strength of the skin, wherein the process utilizes the following steps:
Solubilization of rye fibers and/or seeds and/or bran in water,
Simultaneous or successive enzymatic hydrolysis or hydrolyses,
Separation of soluble and insoluble phases by filtration, centrifuging, decanting,
Treatment of the active fraction, and
Sterilizing filtration.
   The product obtained by the process, cosmetic compositions containing the product, and method of using the product are also disclosed.

This invention relates to a process for obtaining an active ingredient that is obtained from rye fibers and/or seeds and/or bran in order to increase the mechanical strength of the skin and to fight in particular against the appearance of signs of cutaneous aging.

The invention also relates to the active ingredient that can be obtained by this process and the associated cosmetic compositions.

In our societies, great importance is given to cutaneous aging, in particular to aesthetic and psychosocial aspects that are derived therefrom.

The cutaneous aging results from various changes of the dermis caused by factors that are both genetic and environmental. It is manifested in particular by the loss of the mechanical strength and the lifting properties of the dermis. The skin then has a tendency to extend under the influence of its own weight, thus causing surface deformations, and the formation of unsightly wrinkles and folds in particular at the eyelids and the low portion of the facial plane.

To appear younger and to firm up their skin, many people wish to minimize these directly visible unaesthetic physical modifications.

This is what the active ingredient according to this invention proposes by stimulating the natural cutaneous devices involved in the mechanical strength of the skin and by renormalizing its natural lifting properties.

The dermis is a connective tissue for support that essentially consists of fibroblasts and a microfibrillary network of collagen, elastin and proteoglycans that form the extracellular matrix.

The extracellular matrix is the substrate for adhesion of fibroblasts and the mechanical support of the tissue. In the case of pressure on the surface of the skin, the fibroblasts detect the mechanical stresses transmitted by mechanoreceptors, responsible for the junction between the fibroblasts and the extracellular matrix, and in particular respond by stimulating the synthesis of collagen and by inhibiting the production of metalloproteinases.

The mechanoreceptors consist of integrins, proteins of the cellular membrane whose intracellular portion is combined with a focal adhesion complex that consists of a set of structural and signaling proteins, such as vinculin. Stress fibers, including alpha-smooth muscle actin (α-SMA), retractile elements of the cytoskeleton of the fibroblast, are attached to this complex. These fibers, produced by the fibroblasts to raise their retractile properties, ensure the maturation of the mechanoreceptors and allow a more effective transmission of the mechanical signal.

The synergistic activity of the mechanoreceptors and the α-SMA fibers ensures the perpetual equilibrium between the state of contraction and the state of cutaneous relaxation and thus regulates the natural lifting properties of the skin.

During the cutaneous aging, this equilibrium is disrupted leading to the loss of mechanical properties of the dermis.

The fibroblasts that are obtained from aged or photoexposed skin show in particular a reduction of the α2β1 integrins and the vinculin, and therefore a reduction of the formation of mechanoreceptors, which leads to the loss of the contact between the fibroblasts and the extracellular matrix.

Actually, this invention has as its object to promote the formation of mechanoreceptors by stimulating the synthesis of integrins and vinculin, marker protein of focal adhesions, and thus to restore the adhesion capabilities of fibroblasts.

In addition, the fibroblasts that are obtained from aged skin are also characterized by short and disrupted stress fibers in contrast to the fibroblasts of young skin. This disruption of the cytoskeleton brings about a reduction of the retractile forces and consequently a reduced cutaneous mechanical strength.

This is why this invention also proposes increasing the mechanical strength of the skin by stimulating the synthesis of the α-SMA fibers that are responsible for the generation of the retractile forces of the fibroblasts.

The active ingredient according to this invention therefore restores the strength of the skin and its natural lifting properties both:
By promoting the formation of mechanoreceptors by stimulation of the synthesis of the α2β1 integrin and the vinculin, and
By increasing the mechanical strength of the skin by stimulation of the synthesis of α-SMA.

Thus, by doping the cutaneous natural equipment involved in the mechanical strength of the skin and by renormalizing its natural lifting properties, the active ingredient according to the invention firms the cellular tissue. Advantageously, it makes it possible to reinforce the mechanical strength of the skin by increasing the tonicity and the tension of the skin and reducing the wrinkles and fine lines in particular as far as crow's feet and the nasion furrow are concerned.

This invention is now described in detail to make it possible to better grasp the results that are obtained and that are grouped in tables.

### I/Process for Obtaining the Active Ingredient According to the Invention:

The process according to this invention comprises the series of the following stages:

Solubilization of rye fibers and/or seeds and/or bran in water,

Simultaneous or successive enzymatic hydrolysis or hydrolyses,

Separation of soluble and insoluble phases by filtration, centrifuging, decanting,

Treatment of the active fraction, and

Sterilizing filtration.

According to a preferred embodiment of the invention, the process comprises the series of the following stages:

Solubilization of rye seeds and/or bran in water,

Simultaneous or successive enzymatic hydrolysis or hydrolyses,

Separation of soluble and insoluble phases by filtration, centrifuging, decanting,

Heat treatment,

Purification of the active fraction by filtration, and

Sterilizing filtration.

### II/Characterization of the Active Ingredient According to the Invention

II.1/Dry Materials
The level of dry materials is measured by passing a sample of given initial weight into the oven at 105° C. until a constant weight is obtained.

The level of dry materials is between 17 and 210 g/l, more particularly between 50 and 70 g/l.

II.2/Measurement of the pH
The pH that is measured by the potentiometric method at ambient temperature leads to values encompassed between 6 and 9, more particularly between 7 and 8.

II.3/Determination of the Total Sugar Content
The method of DUBOIS (DUBOIS, M. & al. [1956], Analytical Chemistry, 28, No. 3, pp. 350-356) is used.
In the presence of concentrated sulfuric acid and phenol, the reducing sugars provide a yellow-orange compound.
Starting from a standard range, it is possible to determine the total sugar level of a sample.
The total sugar level of the active ingredient according to this invention is 14 to 195 g/l, preferably 45 to 65 g/l.

II.4/Characterization of Carbohydrates

a. Metering of Simple Sugars
The level of simple sugars is divided into 96.4% glucose, 2.6% xylose and 1.0% arabinose.

b. Degree of Polymerization
The table below shows that the glucidic fraction of the active ingredient according to this invention comprises glucose, xylose and arabinose essentially in the form of disaccharides, pentasaccharides and oligosaccharides.

| | Degree of | Level of |
|---|---|---|
| | Polymerization | Carbohydrates |
| Monosaccharides | 1 | 4.8% |
| Disaccharides | 2 | 25.0% |
| Oligosaccharides | 3 | 8.5% |
| Oligosaccharides | 4 | 3.9% |
| Oligosaccharides | 5 | 19.1% |
| Oligosaccharides | 6 | 7.0% |
| Oligosaccharides and | ≧7 | 27.6% |
| Polysaccharides | | |

II.5/Characterization of the Phenolic Compounds
The characterization and the quantification of the phenolic compounds of the active ingredient that is obtained according to the invention are achieved by HPLC (High Performance Liquid Chromatography).
The chromatogram of the active ingredient that is obtained according to the invention shows tie presence of hydroxybenzoic compounds and hydroxycinnamic compounds, whereby the proportions are approximately on the order of the values that are provided in the following table:

| Identified Phenolic Compounds | Concentration |
|---|---|
| Hydroxybenzoic | 16.1% |
| Hydroxycinnamic | 83.9% |
| | Including 71.4% Ferulic Acid and |
| | 10.1% p-Coumaric Acid |

II.6/Identification of the Active Fraction
For the purpose of identifying the active fraction or fractions, the sugars of the active ingredient that is obtained according to the invention are fractionated by gel filtration chromatography.
The effectiveness of the fraction that is obtained is evaluated by the expression of the mRNA of the vinculin by quantitative PCR (Polymerase Chain Reaction or Polymerase Chain Amplification).
The analysis of the results shows that the effectiveness of the active ingredient that is obtained according to the invention resides in an oligosaccharide fraction that is high in arabinoxylan and glucose.

### III Evaluation of the Effect of the Active Ingredient

III.1/Evaluation of the Effect on the Synthesis of Proteins of the Mechanoreceptor
The object of this study is to evaluate the effect of the active ingredient that is obtained according to the invention on the expression of proteins of the mechanoreceptor, namely:
   The integrins of type α2β1, and
   The vinculin, marker of focal adhesions.
The study is carried out by quantitative PCR on normal human fibroblasts, compared to a model of aged human fibroblasts.
At J1, the normal and aged human fibroblasts are inoculated and incubated at 37° C.
At J3, the cells are treated. They are cultivated in the presence of the active ingredient that is obtained according to the invention and metered at 0.25% or TGF-β1 at 1 ng/ml, reference molecule.

At J4, the cells are recovered and the total RNA are extracted.

The RNA are reverse transcripts and the complementary DNA that are obtained are analyzed by the quantitative PCR technique.

a. Effect on the Synthesis of the Integrins α2β1 on Normal Human Fibroblasts and Aged Human Fibroblasts
The results that are obtained relating to the expression of messenger RNA (mRNA) of the integrins α2β1 are expressed in terms of percentage in the following table:

mRNA Level of the
Integrins α2β1 (%)

| | Normal Human | Aged Human |
|---|---|---|
| | Fibroblasts | Fibroblasts |
| Control (Untreated) | 100 | 84 |
| TGF-β1 at 1 ng/ml | 161 | 150 |
| Active Ingredient According | 120 | 109 |
| to the Invention at 0.25% | | |

It is noted that the expression of the mRNA of the integrins α2β1 by the aged fibroblasts is reduced by 16% relative to the normal human fibroblasts.
The results of the study show that the active ingredient according to the invention and that is metered at 0.25% increases by 20% the expression of the mRNA of the integrins α2β1 by the normal fibroblasts and restores the expression of the mRNA of the integrins α2β1 by the aged fibroblasts.

b. Effect on the Synthesis of the Vinculin on Normal Human Fibroblasts and Aged Human Fibroblasts
The results that are obtained relating to the expression of the mRNA of the vinculin are expressed in terms of percentage in the following table.

mRNA Level of the
Vinculin (%)

| | Normal Human | Aged Human |
|---|---|---|
| | Fibroblasts | Fibroblasts |
| Control (Untreated) | 100 | 81 |
| Active Ingredient According | 128 | 103 |
| to the Invention at 0.25% | | |

It is noted that the expression of the mRNA of the vinculin by the aged fibroblasts is reduced by 19% relative to the normal human fibroblasts.
The active ingredient according to the invention that is metered at 0.25% increases by 28% the expression of the in RNA of the vinculin by the normal fibroblasts and restores the expression of the mRNA of the vinculin by the aged fibroblasts.
The active ingredient according to the invention therefore promotes the formation of the mechanoreceptors by stimulating the synthesis of the integrin α2β1 and the vinculin and thus participates in the increase of the strength of the skin and its natural lifting properties.

III.2/Evaluation of the Effect on the Expression of the Alpha-Smooth Muscle Actin

The object of the study is to evaluate the effect of the active ingredient that is obtained according to the invention on the expression of the alpha-smooth muscle actin (α-SMA), stress fiber involved in the retractile properties of the fibroblasts. This study is made on normal human fibroblasts compared to a model of aged human fibroblasts.

a. Study on Normal Human Fibroblasts and Aged Human Fibroblasts by Spectrofluorimetry
The operating procedure is as follows:
At J1, the normal and aged human fibroblasts are inoculated and incubated at 37° C.
At J3, the cells are treated. They are cultivated in the presence of the active ingredient according to the invention at 0.25% or 0.50% or of TGF-β1 at 1 ng/ml.
At J5, the cells are treated as at J3.
At J8, the immunomarking is done in particular with an anti-α-SMA primary antibody. The fluorescence is quantified with a fluorimeter.
The results that are obtained, expressed in terms of percentage of α-SMA expressed, are presented in the following table:

Level of α-SMA (%)

| | Normal | Aged |
|---|---|---|
| | Human | Human |
| | Fibroblasts | Fibroblasts |
| Control (Untreated) | 100 | 40 |
| TGF-β1 at 1 ng/ml | 194 | 140 |
| Active Ingredient According | 134 | 65 |
| to the Invention at 0.25% | | |
| Active Ingredient According | 162 | 72 |
| to the Invention at 0.50% | | |

These results show that the active ingredient that is obtained according to the invention and that is tested at 0.50% on normal fibroblasts increases the level of the α-SMA by 62% relative to the control and stimulates the level of the α-SMA on aged fibroblasts.

b. Study on Normal Human Fibroblasts and Aged Human Fibroblasts by Immunocytology
The operating procedure is as follows:
At J1, the human fibroblasts are inoculated in a complete culture medium.
At J3, the cells are treated. The normal and aged human fibroblasts are treated with the active ingredient according to the invention at 0.10% or with the TGF-β1 at 0.50 ng/ml in the complete culture medium.
At J5, the cells are treated as at J3.
At J8, the immunocytological marking of the α-SMA is carried out, then an immunomarking is carried out in particular with an anti-α-SMA primary antibody.
The results are visualized on a microscope that is linked to an image analysis system. These immunohistochemical results being qualitative, four expression levels of the α-SMA have been defined:
   Very low detection of immunoreactivity +
   Low detection of immunoreactivity ++
   Medium detection of immunoreactivity +++
   High detection of immunoreactivity ++++
The results that are obtained are presented in the following table:

Expression of the α-SMA

| | Normal | |
|---|---|---|
| | Human | Aged Human |
| | Fibroblasts | Fibroblasts |
| Control (Untreated) | +++ | + |
| Active Ingredient According to | ++++ | ++ |
| the Invention at 0.10% | | |

It is noted that the active ingredient that is obtained according to the invention and that is metered at 0.10% increases the expression of the α-SMA on fibroblasts.
The active ingredient that is obtained according to the invention therefore increases the mechanical strength of the skin by stimulating the synthesis of α-SMA.

III.3/Evaluation of the Effect on the Expression of Collagen I
The object of this study is to evaluate the effect of the active ingredient that is obtained according to the invention on the synthesis of collagen I on fibroblasts.
The study is carried out by quantitative PCR on normal human fibroblasts compared to a model of aged human fibroblasts.
At J1, the normal and aged human fibroblasts are inoculated at 37° C.
At J3, the cells are treated. They are cultivated in the presence of the active ingredient that is obtained according to the invention and that is metered at 0.10% or 0.25%.
At J4, the cells are recovered, and the total RNA is extracted.
The RNA are reverse transcripts and the complementary DNA obtained are analyzed by the quantitative PCR technique.
The results that are obtained that relate to the expression of messenger RNA (mRNA) of collagen I are expressed in terms of percentage in the following table:

mRNA Level of
Collage I (%)

| | Normal | |
|---|---|---|
| | Human | Aged Human |
| | Fibroblasts | Fibroblasts |
| Control (Untreated) | 100 | 64 |
| Active Ingredient According | — | 106 |
| to the Invention at 0.10% | | |
| Active Ingredient According | — | 129 |
| to the invention at 0.25% | | |

It is noted that the expression of the mRNA of collagen I by the aged fibroblasts is reduced by 36% relative to the normal human fibroblasts. The results of the study show that the active ingredient according to the invention that is metered at 0.25% increases by 29% the expression of the mRNA of collagen I by the aged fibroblasts.

III.4/Evaluation of the Effect on the Biomechanical Properties of the Skin
The object of this study is to evaluate the effectiveness of the active ingredient that is obtained according to the invention and that is formulated with 4% counter-placebo emulsion on the biomechanical properties of the skin.
The study is carried out on 20 healthy female volunteers between 39 and 70 years of age.
The measurements are made on the face using a Cutometer. The Cutometer is a measuring device that sucks in the Skin and makes it possible to calculate various parameters that are characteristic of the cutaneous mechanical properties:
   The tonicity of the skin is assessed by the parameter X that corresponds to the tonicity or elastic retraction: if X decreases, the tonicity increases.
   The tensor effect is assessed by the parameters Uf and Ue; if Uf decreases, the skin is less extendable and therefore more stretched, and if Ue decreases, the skin is less flexible and therefore also more stretched.
The study is carried out according to the following operating procedure.
At J0, two symmetrical cutaneous zones are determined on the face of each volunteer, one intended for the placebo, the other for the emulsion that contains the active ingredient that is obtained, and the biomechanical properties of the skin are measured in these two zones.
Between J0 and J27, the active ingredient and the placebo are administered twice per day.
At J28, the mechanical properties of the skin are measured on the same zones as at J0.
Between J28 and J55, the active ingredient and the placebo are administered twice per day.
At J56, the mechanical properties of the skin are measured on the zones that are being studied.

a. Tonicity of the Skin
The results that are obtained for the parameter −X are as follows:

| | | | | Active Ingredient | | |
|---|---|---|---|---|---|---|
| | | | | According to the | | |
| | Placebo | | | Invention | | |
| | J0 | J28 | J56 | J0 | J28 | J56 |
| Average of the | 0.638 | 0.793 | 0.618 | 0.754 | 0.810 | 0.594 |
| Volunteers | | | | | | |

| | | |
|---|---|---|
| Variation (Active Ingredient/Placebo in %) | +11 | +19 |

These results show that after 56 days of twice-daily administrations and in comparison to the placebo, the active ingredient that is obtained according to the invention increases the parameter X that is characteristic of the tonicity of the skin by 19%.

b. Tension of the Skin
The results for the parameter −Uf are expressed in the following table:

| | | | | Active Ingredient | | |
|---|---|---|---|---|---|---|
| | | | | According to the | | |
| | Placebo | | | Invention | | |
| | J0 | J28 | J56 | J0 | J28 | J56 |
| Average of the | 1.010 | 1.169 | 0.936 | 1.150 | 1.208 | 0.914 |
| Volunteers | | | | | | |

| | | |
|---|---|---|
| Variation (Active Ingredient/Placebo in %) | +9 | +16 |

Relative to the parameter −Ue, the results are as follows:

| | | | | Active Ingredient | | |
|---|---|---|---|---|---|---|
| | | | | According to the | | |
| | Placebo | | | Invention | | |
| | J0 | J28 | J56 | J0 | J28 | J56 |
| Average of the | 0.779 | 0.930 | 0.725 | 0.926 | 0.968 | 0.709 |
| Volunteers | | | | | | |

| | | |
|---|---|---|
| Variation (Active Ingredient/Placebo in %) | +10 | +19 |

It is noted that the active ingredient that is obtained according to the invention and that is tested with 4% emulsion increases the parameters −Uf by 16% and −Ue by 19%, parameters that are representative of the tension of the skin.
The active ingredient that is obtained according to the invention therefore improves the biomechanical properties of the skin: it increases the tonicity and the cutaneous tension.

III.5/Study of the Anti-Wrinkle Properties
The object of this study is to quantify the anti-wrinkle effectiveness of the active ingredient that is obtained according to the invention and that is formulated with 4% counter-placebo emulsion.
It is carried out on 20 healthy female volunteers between 39 and 70 years of age.
The anti-wrinkle effectiveness is measured by means of silicone-containing impressions made of the crowns feet and the nasion furrow of the volunteers. The analysis of these impressions using a profilometer equipped with an image analyzer makes it possible to obtain three parameters: the number of wrinkles, the total wrinkled surface area, and the total length of the wrinkles.

a. Regarding Crow's Feet
The study is carried out according to the procedure below.
At J0, two symmetrical cutaneous zones at the crow's feet are determined: one intended to be treated by the placebo, the other by the active ingredient, and impressions are taken in these two zones.
Between J0 and J27, the active ingredient and the placebo are administered twice per day.
At J28, the impressions are taken in the two zones that are being studied.
Between J28 and J55, the active ingredient and the placebo are administered twice per day.
At J56, the impressions are taken in the two zones that are being studied.
The results that are obtained for the active ingredient relative to those obtained for the placebo are expressed in terms of percentage in the following table:

Variation/Placebo
(%)

| | At J28 | At J56 |
|---|---|---|
| Number of Wrinkles | +1 | −14 |
| Total Wrinkled Surface Area | −1 | −16 |
| Total Length | +1 | −16 |

It is noted that after 56 days of twice-daily administrations in comparison to the placebo, the active ingredient that is obtained according to the invention and that is formulated with 4% emulsion reduces the number of wrinkles, the total wrinkled surface area and the total length of the wrinkles all at the same time.

b. Regarding the Nasion Furrow
The study is carried out according to the following operating procedure.
At J0, two symmetrical cutaneous zones at the nasion furrow are determined one intended to be treated by the placebo and the other by the active ingredient, and impressions are taken in these two zones.
Between J0 and J27, the active ingredient and the placebo are administered twice per day.
At J28, the impressions are taken in the two zones that are being studied.
Between J28 and J55, the active ingredient and the placebo are administered twice per day.
At J56, the impressions are taken in the two zones that are being studied.
The results that are obtained for the active ingredient in comparison to those obtained for the placebo are expressed in terms of percentage in the following table:

Variation/Placebo
(%)

| | At J28 | At J56 |
|---|---|---|
| Number of Wrinkles | −17 | −23 |
| Total Wrinkled Surface Area | −9 | −26 |
| Total Length | −13 | −27 |

It is noted that after 56 days of twice-daily administrations in comparison to the placebo, the active ingredient that is obtained according to the invention and that is formulated with 4% emulsion reduces the number of wrinkles by 23%, the total wrinkled surface area by 26%, and the total length of wrinkles by 27%.
The active ingredient that is obtained according to the invention therefore has lifting properties and an anti-wrinkle effect.

III.6/Study of Smoothing Properties
The object of this study is to quantify the smoothing effectiveness of the active ingredient that is obtained according to the invention and that is formulated with 4% counter-placebo emulsion.
The study is carried out on 20 healthy female volunteers of between 39 and 70 years of age.
The smoothing effectiveness is measured by means of silicone-containing impressions made of the crow's feet of the volunteers. The analysis of these impressions using a profilometer that is equipped with an image analyzer makes it possible to obtain two parameters: an index Ra of average roughness and an index Rz of maximum roughness.
The study is carried out according to the procedure below.
At J0, two symmetrical cutaneous zones at the crow's feet are determined: one intended to be treated by the placebo and the other by the active ingredient, and the impressions are taken in these two zones.
Between J0 and J27, the active ingredient and the placebo are administered twice per day.
At J28, the impressions are taken in the two zones that are being studied.
Between J28 and J55, the active ingredient and the placebo are administered twice per day.
At J56, the impressions are taken in the two zones that are being studied.
The results that are obtained for the active ingredient relative to those obtained for the placebo are expressed in terms of percentage in the following table:

Variation/Placebo
(%) at J56

| | At J28 | At J56 |
|---|---|---|
| Index of Average Roughness (Ra) | −4.8 | −7.8 |
| Index of Maximum Roughness (Rz) | −4.0 | −5.0 |

It is noted that after 56 days of twice-daily administrations in comparison to the placebo, the active ingredient that is obtained according to the invention and that is formulated with 4% emulsion reduces the indices of average and maximum roughness.
The active ingredient that is obtained according to the invention therefore has a smoothing effect.

### IV/Cosmetic Composition Including the Active Ingredient According to the Invention;

This invention also covers the cosmetic compositions that include the active ingredient according to this invention in various galenical forms, in particular gel, solution, emulsion, cream . . . .

It is then suitable to analyze the stability of the galenical forms that include the active ingredient according to the invention in proportions of between 1 and 5%.

The stability is characterized by an absence of precipitation of the active ingredient, an absence of creaming, and an absence of phase shift.

It is possible to cite formulations that have shown a physical stability that includes 5% active ingredient according to the invention.

| | |
|---|---|
| Clear Gel: | Carbopol: 0.5% with triethanolamine: sufficient |
| | quantity for pH = 6.5 |
| | Phenonip: 0.7% |
| | Active ingredient: 5.0% |
| | Water: 93.8% |
| Opaque Gel: | Sepigel 305: 2.0% |
| | Phenonip: 0.7% |
| | Active ingredient: 5.0% |
| | Water: 92.3% |
| Emulsified Gel: | Montanov 202: 3.0% |
| | Isopropyl palmitate: 12.0% |
| | Phenonip: 0.7% |
| | Viscolam AT 64: 2.0% |
| | Active ingredient: 5.0% |
| | Water: 77.3% |
| Non-Ionic Emulsion: | Montanov 202: 3.0% |
| | Simulsol 165: 2.0% |
| | Isopropyl palmitate: 20.0% |
| | Phenonip: 0.7% |
| | Active ingredient: 5.0% |
| | Water: 69.3% |
| Anionic Emulsion: | Stearic acid: 7.0% |
| | Triethanolamine: 3.5% |
| | Isopropyl palmitate: 20.0% |
| | Phenonip: 0.7% |
| | Active ingredient: 5.0% |
| | Water: 63.8% |
| Cationic Emulsion: | Quaternium-82: 5.0% |
| | Cetylic alcohol: 3.0% |
| | Isopropyl palmitate: 15.0% |
| | Phenonip: 0.7% |
| | Active ingredient: 5.0% |
| | Water: 71.3% |

In addition, tests have shown the compatibility of the active ingredient with the raw materials used in cosmetics.

1-11. (canceled)

12. Process for obtaining an active ingredient for increasing the mechanical strength of the skin, characterized in that it comprises the following stages:
Solubilization of rye fibers and/or seeds and/or bran in water,
Simultaneous or successive enzymatic hydrolysis or hydrolyses,
Separation of soluble and insoluble phases by filtration, centrifuging, decanting,
Treatment of the active fraction, and
Sterilizing filtration.

13. The process for obtaining an active ingredient for increasing the mechanical strength of the skin according to claim 12, wherein the solubilization stage consists in solubilizing rye seeds and/or bran.

14. The process for obtaining an active ingredient for increasing the mechanical strength of the skin according to claim 12, wherein the treatment of the active fraction is carried out by heat treatment followed by a purification by filtration, ultrafiltration, reverse osmosis or nanofiltration.

15. An active ingredient that is obtained by the implementation of the process according to claim 12, characterized by the following parameters:
Level of dry materials of between 17 and 210 g/l,
pH of between 6.0 and 9.0, and
Total sugar content of between 14 and 195 g/l.

16. The active ingredient according to claim 15, wherein the following parameters:
Level of dry materials of between 50 and 70 g/l,
pH of between 7.0 and 8.0,
Protein content of between 45 and 65 g/l,
Presence of carbohydrates in the form of glucose, xylose and arabinose, and
Presence of phenolic compounds in the form of hydroxybenzoic and hydroxycinnamic compounds.

17. A method for enhancing cutaneous mechanical strength comprising administering an effective amount of the active ingredient according to claim 15.

18. A method for increasing tension and tonicity of skin, comprising administering an effective amount of the active ingredient according to claim 15.

19. A method for providing an anti-wrinkle effect, comprising administering an effective amount of the active ingredient according to claim 15.

20. A method for promoting the formation of the mechanoreceptors of the fibroblasts of the dermis, comprising administering an effective amount of the active ingredient according to claim 15.

21. A method for stimulating the synthesis of the alpha-smooth actin fibers, comprising administering an effective amount of the active ingredient according to claim 15.

22. A cosmetic composition that includes the active ingredient according to claim 15, wherein said cosmetic is a clear gel, an opaque gel, an emulsified gel, a non-ionic emulsion, an anionic emulsion or a cationic emulsion.
